(19) United States
(12) Patent Application Publication
(10) Pub. No.: US 2013/0005749 A1
(43) Pub. Date: Jan. 3, 2013

(54) NOVEL FORMULATION AND TREATMENT METHODS

(75) Inventors: Timothy Henri Jeremy Florin, Buranda (AU); Yunmei Song, Modbury (AU); Xianling Luo, North Adelaide (AU)

(73) Assignee: The University of Queensland, St. Lucia (AU)

(21) Appl. No.: 13/492,524

(22) Filed: Jun. 8, 2012

(30) Foreign Application Priority Data
Jun. 10, 2011 (AU) 2011902301

(51) Int. Cl. A61K 31/52; A61P 37/06; A61P 35/00; C07D 473/38; A61P 29/00

(52) U.S. Cl. 514/263.37; 544/276

(57) ABSTRACT

The invention relates to extended release compositions and formulations comprising 6-thioguanine (6-TG), and methods for treating diseases or conditions responsive to 6-TG.

### FIELD OF THE INVENTION

The present invention relates generally to novel formulations of 6-thioguanine and their use in improved treatment methods.

### BACKGROUND OF THE INVENTION

The closely related thiopurine compounds 6-mercaptopurine or “6-MP” (3,7-dihydropurine-6-thione) and azathioprine or “AZA” (6-(3-methyl-5-nitroimidazol-4-yl)sulfanyl-7H-purine) are pro-drugs which use the salvage pathway of purine metabolism to form the principle active drug, 6-thioguanosine-triphosphate (“6-TGTP”).

AZA and 6-MP are used in treating a number of diseases and conditions, most notably inflammatory conditions (especially inflammatory bowel diseases), cancers (especially childhood leukemia), autoimmune conditions, and post-transplant immunosuppresion. They are a cornerstone of maintenance therapy for inflammatory bowel disease despite (i) the long time-to-onset of clinical activity (i.e., the pharmacodynamic action), for example 2-4 months in inflammatory bowel disease although steady state levels of active drug are reached within 4 weeks, and (ii) the relatively fine line between the desired clinical end-point of controlled immunosuppression and the side-effect of leukopenia.

Metabolism of AZA or 6-MP by some cells in the body, including liver cells and white blood cells, leads to the active drug, thioguanine nucleotides (principally 6-TGTP, and to a lesser extent 6-TGMP or 6-TGDP), but also methylated metabolites, including methyl-6MP, methyl-6MP riboside, methyl-thioIMP, and methyl-thioIMP riboside and an imidazole metabolite of AZA. High levels of these metabolites are associated with a number of undesirable side-effects, including hepatotoxicity, pancreatitis, myelosuppression (beyond that of an immunosuppressive effect) as described in (Dubinsky et al. 2002; McGovern et al. 2002; and Hindorf et al. 2006).

These side-effects are serious in a large percentage of patients currently prescribed either 6-MP or AZA, and approximately 40% of patients stop therapy after one year due either to these side-effects or non-responsiveness to the therapy (Ansari et al. 2008).

Another thiopurine compound, 6-thioguanine or “6-TG” (2-amino-6,7-dihydro-3H-purine-6-thione), is sometimes prescribed as an alternative in the situation when 6-MP or AZA therapy has been tried and failed. 6-TG is metabolised more directly by the enzyme hypoxanthine-guanine phosphoribosyltransferase (HPRT) to 6-TGTP, i.e., the same principal active compound formed by metabolism of 6-MP or AZA, but the pathway to reach this active product is different. Specifically, 6-TG is metabolised to the active drug with fewer steps than 6-MP or AZA. As such, 6-TG has a clinically significant faster onset of action than 6-MP or AZA. Furthermore, the less desirable methylated metabolites produced during metabolism of 6-MP or AZA are not produced when the body metabolises 6-TG.

On this basis it would appear that 6-TG should be a preferred drug candidate over 6-MP or AZA. However, currently this is not the case because 6-TG treatment also leads to a particular and dreaded adverse side-effect: sinusoidal obstructive syndrome or SOS (also referred to as veno-occlusive disease or VOD or the related nodular regenerative hyperplasia or NRH). Studies to date have indicated that SOS (or VOD/NRH) is a side-effect strongly associated with 6-TG and not 6-MP or AZA. For example, a study of acute childhood leukemia cases demonstrated a high prevalence of VOD in patients treated with 6-TG that was not observed in the patients treated with 6-MP (Stork et al. 2010). The SOS side-effect has relegated 6-TG to an experimental treatment option in most parts of the world (Seinen et al. 2010). It must be noted that the study in Stork et al. 2010 (and others) demonstrated 6-TG to be equivalent or even more clinically effective in treating the disease than 6-MP.

In work leading up to the present invention, the inventors studied the effect of 6-TG treatment on HPRT^{-/-} mice, i.e., mice lacking the enzyme to convert 6-TG to the principal active nucleotide drug 6-TGTP (and/or 6-TGMP or 6-TGDP). Surprisingly, they discovered that these mice did not exhibit the SOS side-effect associated with 6-TG. Instead, the results showed that it is more likely to be the active drug itself, or possibly a metabolite of the nucleotide, and not 6-TG or a metabolite thereof (such as methyl-6TG) that is not the active drug, that is responsible for the undesirable side-effect of SOS associated with 6-TG. Although 6-MP and AZA are ultimately converted to thioguanine nucleotides, the inventors postulate that SOS is strongly associated with 6-TG and not 6-MP or AZA because 6-TG is more readily converted to the active drug (the thioguanine nucleotides 6-TGTP, 6-TGMP, and/or 6-TGDP) with the result that higher concentrations of thioguanine nucleotides occur in the liver to cause the off-target side-effect of SOS, upon administration of 6-TG compared to 6-MP or AZA.

To investigate this discovery further, the inventors administered a large dose of 6-TG to mice orally and to a separate group of mice by intraperitoneal injection. Those mice who received the 6-TG orally exhibited the SOS side-effect associated with 6-TG, those findings being consistent with previous studies. However, the mice that received the same amount of 6-TG into the peritoneal cavity did not exhibit the SOS side-effect associated with 6-TG. Injection into the peritoneal cavity avoids the portal circulation.

Accordingly, the inventors had thus determined that upon administration of 6-TG, the 6-TG is metabolised into the active drug 6-TGTP (and/or 6-TGMP or 6-TGDP) which acts on target cells (e.g., activated T-lymphocytes) to induce a therapeutic effect, but also acts on off-target cells (e.g., liver cells) to induce the adverse side-effects (e.g., SOS).

The inventors then administered to mice the same total dose of 6-TG as the large dose previously administered, but in two separate dosages (halved), by oral administration. These mice exhibited substantially less of the undesirable side-effect SOS. Other divided dosing (or split dosing) options were also explored, with similar results obtained.

These results and subsequent work by the inventors has enabled them to define a therapeutic protocol for administering 6-TG, including extended-release formulations or divided-dosing treatment regimes, that provide the active drug in an amount required to induce effective treatment to the target cells in the body (e.g., T-lymphocytes) but without providing a toxic level of the active drug to off-target cells in the body (e.g., liver cells) that occurs in conventional therapy. Thus these protocols achieve the desirable effects of 6-TG therapy (e.g., treatment of the disease or condition, faster therapeutic effect compared to 6-MP or AZA, none of the 6-MP or AZA undesirable metabolites), without eliciting the undesirable side-effects observed with 6-TG therapies to date (e.g., SOS).

### SUMMARY OF THE INVENTION

Accordingly, the present invention relates to therapeutic protocols for the treatment of diseases or conditions that respond to 6-thioguanine (6-TG) therapy. In particular, the therapeutic protocol contemplates compositions and methods that can be used to treat the disease or condition with reduced or no adverse 6-TG side-effects compared to conventional 6-TG therapy (e.g., bolus oral administration) of comparable (e.g., same, similar, or substantially similar) 6-TG dosage (also referred to herein as “optimal therapeutic protocol”). In particular, the therapeutic protocol avoids the SOS side-effect of conventional 6-TG therapy. In specific embodiments, it is considered that the optimal therapeutic protocol comprises a total daily dose of 6-TG of between 0.3 mg/kg body weight to 1.5 mg/kg body weight, where administered orally, where the dose is not administered as a bolus administration. Suitably, the 6-TG is administered in an extended-release formulation or as a divided dose. In other specific embodiments, it is considered that the optimal therapeutic protocol comprises a total daily dose of 6-TG of between 0.3 mg/kg body weight to 1.5 mg/kg body weight, where administered by a route that avoids a bolus dose directed to the liver (i.e., avoids the first-pass effect). Suitably, the 6-TG is administered by an intraperitoneal, buccal, transdermal, intranasal, inhalation or sublingual administration route.

Thus in a first aspect, the present invention provides a composition comprising an extended-release formulation of 6-TG. Suitably, the composition is formulated to provide a dose of 6-TG of between 0.3 mg/kg body weight/day to 1.5 mg/kg body weight/day. Suitably, the composition is formulated for oral administration, or other forms of administration amenable to extended release, or avoidance of the first pass hepatoportal circulation, such as transdermal patch or implant. Suitably, the composition is for treating a disease or condition responsive to 6-TG.

In another aspect, the present invention provides a method for treating a disease or condition that responds to 6-TG in an individual in need thereof, the method comprising administering to the individual a total dose of 6-TG, wherein the method avoids or at least reduces the 6-TG side-effects observed when a comparable (e.g., same, similar, or substantially similar) total dose of 6-TG is administered by way of conventional 6-TG therapy (e.g., orally in bolus immediate release form). Suitably, the total dose of 6-TG comprises between about 0.3 mg/kg body weight/day to 1.5 mg/kg body weight/day. However, for some disease states, or in certain metabolic contexts, higher or lower doses may be required.

In some embodiments, the method comprises oral, intraperitoneal, buccal, sublingual, transdermal or intranasal administration. In view of the cytoxicity of the active agent, 6-TG, care needs to be taken with any administration route to guard against unacceptable off-target activity.

In some embodiments, the method comprises administering a composition comprising an extended-release formulation of 6-TG, for example the method may comprise administering a composition of the present invention.

In other embodiments, the method comprises an administration regime that comprises administering more than one composition comprising 6-TG, wherein the total dose of 6-TG is divided into the compositions, wherein the compositions are not administered at the same time (e.g., administered at different times). This administration regime may be considered “divided dosing” or “split dosing”. In this method, each of the compositions may be the same or different and the compositions may comprise extended-release formulations of 6-TG or other formulations of 6-TG, such as bolus or immediate release formulations, or mixtures thereof.

In some embodiments, the method further comprises administration of an additional active agent (other than 6-TG) for treating a disease or condition responsive to 6-TG, including an active agent selected from the group consisting of: mesalazine (or 5-aminosalicylic acid), balsalazide, sulfasalazine, a corticosteroid, a cyclosporine compound, an anti-TNF-α compound. The additional active agent may be administered in the same composition or compositions, or in a different composition or composition to the composition(s) comprising 6-TG. The additional active agent may be administered at the same time or at a different time to the composition(s) comprising 6-TG. The present invention includes combinations, kits or commercial packages comprising the 6-TG composition and a composition comprising the additional active agent. Such kits or commercial packages may include instructions for use. In some cases it may be appropriate to provide a composition comprising 6-TG and the other active agent.

In yet another aspect, the present invention provides the use of 6-TG in the manufacture of a composition (e.g., a medicament), wherein the composition is formulated so as to treat a disease or condition that responds to 6-TG with reduced or no adverse 6-TG side-effects compared to conventional 6-TG therapy (e.g., bolus immediate release oral administration) of comparable (e.g., same, similar, or substantially similar) 6-TG dosage. In some embodiments, the composition comprises an extended-release formulation of 6-TG. Suitably the composition may be formulated so as provide a daily dose of 6-TG to the individual of between 0.3 mg/kg body weight to 1.5 mg/kg body weight. Suitably, the composition is formulated for oral, intraperitoneal, buccal, sublingual, transdermal or intranasal administration.

### BRIEF DESCRIPTION OF THE DRAWINGS

FIG. 1 shows the results of Example 1 where SOS (VOD) was only observed only in the mice treated with 6-TG, and not those treated with 6-MP or their methylated bases, as discussed in Example 1.

FIG. 2 shows the results of the 14 day experiment conducted in Example 2. The results are shown as box and whisker plots (median, quartiles and range) where * P≦0.05, ** P≦0.01, *** P≦0.005, Mann Whitney non-parametric tests. On the left hand side of the graph in FIG. 2, the results for the C57BL/6 mice are shown and on the right hand side of the graph in FIG. 2, the results for the HPRT^{-/-} C57BL/6 mice are shown. Dose of 6-TG administered is indicated on the x-axis and blinded histological score on the y-axis (total possible VOD score is 240). VOD was observed in C57BL/6 mice administered 1 and 2.5 mg/kg body weight while VOD was not observed in any of the HPRT^{-/-} C57BL/6 mice, including those given 6-TG dosages of 1 mg/kg body weight and 2.5 mg/kg body weight.

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, the term “about” refers to a quantity, level, concentration, value, size, or amount that varies by as much as 30%, 20%, or 10% or even as much as 9%, 8%, 7%, 6%, 5%, 4%, 3%, 2%, 1%, to a reference quantity, level, concentration, value, size, or amount.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

As used herein, the phrase “diseases or conditions that respond to 6-TG” or “diseases or conditions responsive to 6-TG” and similar refers to any disease or condition for which 6-TG can treat, including treatment of any symptom of the disease or condition. Illustrative examples of such diseases or conditions include cancers, including but not limited to leukemia (especially acute childhood leukemia), inflammatory conditions including but not limited to inflammatory bowel diseases (especially ulcerative colitis and Crohn's disease), autoimmune conditions, eczema, psoriasis, myasthenia gravis, and post-transplant immunosuppresion (including renal transplantation).

The term “extended-release formulation” refers to a formulation that is converted or degraded or metabolised into, or provides a source of, the active component that it contains (e.g., 6-TG) over an extended period of time. Such formulations are also referred to in the art as slow release, controlled release, modified release or sustained release formulations.

The “first-pass effect” is an effect (or phenomenon) of drug metabolism whereby the concentration of a drug (e.g., 6-TG, 6-TGTP, 6-TGMP, 6-TGDP) is greatly reduced before it reaches the systemic circulation. It is the fraction of lost drug during the process of absorption which is generally related to the liver and gut wall.

The terms “individual”, “patient” and “subject” are used interchangeably herein to refer to individuals of human or other animal origin and includes any individual it is desired to examine or treat using the methods of the invention. However, it will be understood that these terms do not imply that symptoms are present. Suitable animals that fall within the scope of the invention include, but are not restricted to, humans, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes, avians, reptiles).

The phase “6-TG side-effects” and similar expressions refers to the adverse or undesirable side-effects caused by 6-TG or a metabolite thereof, including 6-TGTP (and/or 6-TGMP or 6-TGDP), including but not limited to hepatotoxicity, including sinusoidal obstructive syndrome or SOS which is also referred to as veno-occlusive disease or VOD and the closely related nodular regenerative hyperplasia or NRH.

By “pharmaceutically acceptable carrier or diluent” is meant a solid or liquid filler, diluent or encapsulating substance that can be safely used in topical or systemic administration.

The term “sample” as used herein refers to a sample that may be extracted, untreated, treated, diluted or concentrated from a patient. Suitably, the biological sample is a whole blood, serum or plasma sample. The term also refers to isolated fractions of samples from a patient, including an isolated preparation of white blood cells or an isolated preparation of red blood cells.

As used herein, the phrase “target cells” means cells that are intended to be targeted by 6-TG in order to treat the disease or condition response to 6-TG. Illustrative embodiments of target cells include T-lymphocytes, B-lymphocytes and dendritic cells. Similarly, “off target cells” or “non-target cells” and similar refers to those cells not intended to be targeted by 6-TG in order to treat the disease or condition response to 6-TG, but with which the 6-TG will interact and cause an effect. Illustrative embodiments of off target cells include liver cells.

The terms “treat”, “treating” or “treatment” as used herein cover the treatment of a disease or condition responsive to 6-TG in an individual having the disease or condition, and includes: (a) preventing the disease or condition from occurring in the individual, in particular when such individual is predisposed to the condition but has not yet been diagnosed as having it; (b) inhibiting the disease or condition, i.e., arresting its development; (c) relieving the disease or condition, i.e., causing regression of the disease or condition; or (d) relieving the symptoms resulting from the disease or condition, i.e., relieving pain or inflammation without addressing the underlying disease or condition.

### Therapeutic Protocol

The present invention arises from the discovery that the principal active drug produced during 6-TG therapy, namely 6-TGTP (and/or 6-TGMP or 6-TGDP), both interacts with the target cells of the body to elicit the desired therapeutic effect, but is also responsible for the adverse side-effects by virtue of its interaction with the off-target cells (e.g., liver cells). Based on this discovery, the inventors conducted further research, enabling them to define therapeutic protocols for the treatment of diseases or conditions that respond to 6-TG with reduced or no adverse 6-TG side-effects compared to conventional 6-TG therapy of comparable 6-TG dosage (also referred to herein as “optimal therapeutic protocol”).

The optimal therapeutic window is considered to be a dosage that achieves the bioavailability of a conventional 6-TG therapy that is effective in treating a disease or condition responsive to 6-TG. This dosage can be described as having the same (or substantially the same) area under the curve (AUC) of the drug concentration time profile when administered in a bolus dosage. This ensures the same (or a substantially similar) amount of active drug reaches the target cells (e.g., T-lymphocytes) as the amount of conventional 6-TG therapy. However, suitably the optimal therapeutic window comprises a dose-range that does not lead to “over-treatment” of the target cells so as to cause leukopenia. The optimal therapeutic window also achieves reduced or no adverse 6-TG side-effects relative to those observed with the conventional 6-TG therapy by having a Cₘₐₓ in the hepatoportal circulation that is lower than that of the conventional 6-TG therapy. The Cₘₐₓ measurement refers to the maximum (or peak) concentration that a drug achieves in tested area after the drug has been administrated and prior to the administration of a second dose. The lower Cₘₐₓ minimises toxicity (and thus side-effects) by reducing the peak exposure of the off-target cells (e.g., liver cells) to the active drug.

This optimal therapeutic window may be achieved by way of an extended-release formulations or divided dosing (or split-dosing) treatment regimes.

Alternatively, the optimal therapeutic window may be achieved by way of an administration route that avoids a bolus dose of 6-TG directed to the liver (i.e., avoids the first-pass effect), for example, an administration route where delivery is other than through the portal circulation (e.g., intraperitoneal, buccal, sublingual), transdermal or intranasal.

In specific embodiments, the optimal therapeutic protocol comprises a total daily dose of 6-TG of between 0.3 mg/kg body weight to 1.5 mg/kg body weight.

Accordingly, in a first aspect, the present invention provides a composition comprising an extended-release formulation of 6-TG. The composition may be a pharmaceutical composition comprising 6-TG and a pharmaceutically acceptable carrier suitable for providing extended release of 6-TG. Suitably, the composition is formulated to provide a dose of 6-TG of between 0.3 mg/kg body weight/day to 1.5 mg/kg body weight/day. Suitably, the composition is formulated for oral administration, or by another route amendable to slow or extended release of 6-TG. Suitably, the composition is for treating a disease or condition responsive to 6-TG.

In another aspect, the present invention provides a method for treating a disease or condition that responds to 6-TG in an individual in need thereof, the method comprising administering to the individual a total dose of 6-TG, wherein the method avoids or at least reduces the 6-TG side-effects observed when a comparable (e.g., same, similar, or substantially similar) total dose of 6-TG is administered by way of conventional 6-TG therapy (e.g., orally in bolus form). Suitably, the total dose of 6-TG comprises between about 0.3 mg/kg body weight/day to 1.5 mg/kg body weight/day.

In some embodiments, the method comprises oral, intraperitoneal, buccal, sublingual, transdermal or intranasal administration.

In some embodiments, the method comprises administering a composition comprising an extended-release formulation of 6-TG, for example the method may comprise administering a composition of the present invention.

In other embodiments, the method comprises an administration regime that comprises administering more than one composition comprising 6-TG, wherein the total dose of 6-TG is divided into the compositions, wherein the compositions are not administered at the same time (e.g., administered at different times). This administration regime may be considered “divided dosing” or “split dosing”. In this method, each of the compositions may be the same or different and the compositions may comprise extended-release formulations of 6-TG or other formulations of 6-TG such as bolus or immediate release formulations, or mixtures thereof.

In some embodiments, the method further comprises administration of an additional active agent (other than 6-TG) for treating a disease or condition responsive to 6-TG, including an active agent selected from the group consisting of: mesalazine (or 5-aminosalicylic acid), balsalazide, sulfasalazine, a corticosteroid, a cyclosporine compound, an anti-TNF-α compound. The additional active agent may be administered in the same composition or compositions, or in a different composition or composition to the composition(s) comprising 6-TG. The additional active agent may be administered at the same time or at a different time to the composition(s) comprising 6-TG. The present invention indicates combinations, kits or commercial packages comprising the 6-TG composition and a composition comprising the additional active agent. Such kits or commercial packages may include instructions for use. In some cases it may be appropriate to provide a composition comprising 6-TG and the other active agent.

In yet another aspect, the present invention provides the use of 6-TG in the manufacture of a composition (e.g., a medicament), wherein the composition is formulated so as to treat a disease or condition that responds to 6-TG with reduced or no adverse 6-TG side-effects compared to conventional 6-TG therapy (e.g., bolus oral administration) of comparable (e.g., same, similar, or substantially similar) 6-TG dosage. In some embodiments, the composition comprises an extended-release formulation of 6-TG. Suitably the composition may be formulated so as provide a daily dose of 6-TG to the individual of between 0.3 mg/kg body weight to 1.5 mg/kg body weight. Suitably, the composition is formulated for oral, intraperitoneal, buccal, sublingual, transdermal or intranasal administration.

### Extended-Release Formulations

The present invention provides a composition comprising an extended-release formulation of 6-TG. Also provided is a method of a method for treating a disease or condition that responds to 6-TG in an individual in need thereof, the method comprising administering to the individual a composition of the present invention comprising an extended-release formulation of 6-TG.

As used herein, the term “extended-release formulation” refers to a formulation that is converted or degraded or metabolised into, releases, or provides a source of, the pro-drug, drug, or active component that it contains (e.g., 6-TG) over an extended period of time.

In some embodiments, the extended-release formulation provides a steady state profile of 6-TG delivery equivalent to intravenous delivery of 6-TG. Suitably, the extended-release formulation provides zero order kinetics of 6-TG delivery (i.e., a linear delivery with respect to the time of extended release.

Suitably, the extended-release formulations of the present invention, when in oral form, are formulated so as to release or provide 6-TG to the small intestine. In illustrative embodiments, the formulation releases or provides the 6-TG to the small intestine over a period extending from 30 minutes up to 6 hours, suitably the period is no longer than 30 minutes, 45 minutes, 60 minutes, 90 minutes, 2 hours, 2.5 hours, 3 hours, 3.5 hours, 4 hours, 4.5 hours, 5 hours or up to 5.5 hours or any period in between. In illustrative embodiments, the period is between 30 minutes and 6 hours, between 45 minutes and 4 hours, or between 1 hour and 2 hours.

In some embodiments the oral administration formulation provides a 6-TG release of 30±15% in the stomach in the first hour, 60±15% of the 6-TG in the small intestine in the second hour and 80±15% of the 6-TG in the small intestine in the third hour.

Suitably, the composition comprising the extended-release formulation of 6-TG comprises 6-TG, polymer and excipients sufficient to enable 6-TG release into the blood stream, for example the hepatoportal blood stream, at the desired rate of release. In exemplary embodiments, the composition comprises a film coating, or an enteric coat to prevent disintegration of the composition in the stomach.

The extended release formulations may include suitable fillers, for example lactose, sucrose, glucose, mannitol, sorbitol, calcium carbonate, starch, dextrin, cyclodextrin, calcium sulphate dehydrate (or hydrate), tricalcium phosphate, or microcrystalline cellulose.

The formulation may include an extended release agent which may be selected from, for example wax and its derivate, hydrogenated castor oil, hydrogenated soybean phospholipid, shellac, gelatine, sodium alginate, chitosan, fibrin and fibrinogen, pullulan, agar, carrageenan, guar gum, methyl cellulose (MC), ethyl cellulose, hydroxyethyl cellulose, hydroxypropyl cellulose (HPC), hydroxypropyl methyl cellulose (HPMC), carboxymethyl cellulose, carboxymethyl cellulose sodium (NaCMC), croscarmellose sodium, cellulose acetate, hydroxypropyl methylcellulose phthalate, cellulose acetate phthalate, Eudragit, carbopol, polyvinyl alcohol, ethylene-vinylacetate copolymer, ethylene-vinylalcohol copolymer, crospovidone, or pluronic.

The formulation may include a solubilising agent, and these may be selected from, for example cyclodextrin and its derivate, (e.g. hydroxypropyl-beta-cyclodextrin, methyl-beta-cyclodextrin), polyvinylpyrrolidone (PVP), glucose, mannitol, xylitol, sorbitol, carbamide, galactose, sucrose, citric acid, succine acid, polyethylene oxide, polyethylene glycol, poloxamer, sodium lauryl sulphate, sodium cholate, saponins, silica, oil, triglycerides, surfactants such as cremophor, tween, solutol HS, Myrj, phospholipid and span, and alkali materials such as inorganic alkali (e.g. sodium hydroxide and sodium phosphate) and organic alkali (e.g. sodium benzoate and sodium pantothenate)

For tablet formulations a lubricant may be included, which may be selected from, for example, magnesium stearate, stearic acid, calcium stearate, zinc stearate, sterotex, polyethylene glycol, talc, sodium lauryl sulphate, magnesium lauryl sulphate, adipic acid, paraffin and silica gel.

The extended-release formulations of the present invention may be made by any suitable method known in the art, including those described in the latest edition of Controlled drug delivery (Drugs and the pharmaceutical sciences; vol. 29; Marcel Dekker, Inc) and the latest edition of Modified-Release Drug Delivery Technology (Drugs and the pharmaceutical sciences; vol. 126; Marcel Dekker, Inc).

The extended release formulations can be in any suitable form, for example, tablets, capsules, pellets, granules, films and suspensions. Extended release patches, slow release injection, implants and suppositories are also envisaged.

The tablet formulations can be prepared by any suitable tabletting method, although direct compression when possible is particularly advantageous and economical. It is also possible to prepare granules and pellets using any suitable technique, including wet granulation and roller compaction. Tablets, capsules, pellets and granules can also be coated in a suitable extended release agent.

Extended-release of the 6-TG in the compositions of the present invention may be achieved with a formulation that includes disaggregating agents, which on contact with biological fluids encourage disaggregation of the formulation.

The 6-TG may also be blended with one or more polymers, to provide a matrix that is either formed into a particle (small or large), or is coated on an inert particle. The polymers are selected from hydrophilic, hydrophobic or plastic polymers. Hydrophilic polymers are water soluble and hydrate in contact with water to form a hydrogel as they dissolve and swell; hydrophobic polymers do not dissolve but may be subject to erosion as the matrix releases soluble constituents; plastic polymers form insoluble or skeletal matrices but do not erode. Upon exposure to the fluid in the stomach, small intestine and colon, hydrophilic polymers hydrate and form a hydrogel that acts as a barrier to drug release; hydrophobic polymers release drug through diffusion through pores and through erosion. Drug release from plastic matrices is controlled by the rate of liquid penetration and is accelerated by the presence of channel forming agents: soluble components that are added in addition to 6-TG.

The rate of 6-TG release can be decreased by increasing the ratio of polymer to 6-TG, and by increasing the hydrophobicity of the matrix. Suitably, the rate of 6-TG release is decreased so as to achieve release in the small intestine.

The behaviour of some polymers is pH-dependent, and this property may be used to achieve the desired release profile. A polymer will be especially pH-dependent where it contains acidic or basic moieties and these affect the ionisation state. Ionisation can transform a polymer from hydrophobic to hydrophilic, with an accompanying transformation in release properties.

The release of the dissolved drug into, for example, the gastrointestinal (GI) tract may be controlled by a coating on the particle. The coating is typically a polymer or blend of polymers that is relatively stable towards the conditions encountered in the GI tract. In many cases, the coating includes at least one hydrophilic polymer that will swell on contact with fluid in the gut to form a hydrogel barrier that is homogenous and stable to changes that may take place to the underlying matrix. The hydrogel also serves to slow release of dissolved 6-TG. The properties of the surface coating can be pH-dependent depending upon the presence of acidic or basic moieties in the polymer constituents.

A particular feature of extended release formulations is the potential for a burst release of drug to occur immediately following contact of the dosage form with the dissolution fluid. The use of a hydrophilic polymer in the film coating or in the matrix, wherein the hydrophilic polymer forms a hydrogel rapidly after hydration, can significantly reduce the incidence of the burst release phenomenon.

Extended release formulations include a monolithic tablet dosage forms in which one or more drug-polymer matrices provide the core, and an optional surface film coating can provide additional control over drug release. This differs from immediate release (IR) formulations which are designed to disintegrate, dissolve promptly and release a bolus dose of drug.

The matrix may be formed by granulation or direct compression and is heterogeneous to provide porosity.

In particular, a matrix may comprise either or both hydrophilic polymers and hydrophobic polymers in order to achieve the appropriate release profile. Further, one or more of the polymers may swell upon hydration in a manner that may additionally be dependent upon pH, to form a hydrogel that is viscous and gelatinous and thus provides a barrier to drug release. The composition of hydrogel determines its properties, which can thus be manipulated in order to achieve appropriate drug release kinetics.

The film coating provides a diffusion release mechanism where the permeability is often directly related to hydration leading to polymer swelling and the installation of hydrogel dynamics.

Multi-layer tablets have been developed to provide zero order release kinetics for highly soluble drugs. Different layers may also have different release profiles.

At least one combination of matrix and film coating provided in the description below can used to achieve the desired release profile across the different environments encountered during transit through the GI tract. Suitably, the formation provides a release profile in the small intestine.

Microparticulate systems typically consist of spherical particles with diameter 0.05-2.00 mm that are further packaged into a capsule or tablet. Microparticle dosage systems can provide the following benefits for extended release formulations: (1) less dependent on gastric emptying, resulting in less intra/inter individual variability in gastric transit time (sizes less than 2 mm are able to continuously leave stomach even when pylorus is closed); (2) particles are better distributed, avoiding possibility of localized irritation; and (3) drug safety is improved for extended release formulations, as less susceptible to performance failure if damaged. Furthermore it is possible to mix particles with different release profiles to optimise exposure in different regions of gut.

Polymers that are of use in the formation of drug-polymer matrices are as follows: (1) acrylic and methacrylic polymers including hydroxypropyl methacrylates (HPMA) and hydroxyethyl methacrylates (HEMA), as well as N-isopropyl acrylamides; (2) polyethylene oxides (PEO) also known as polyethylene glycols (PEG) and polypropylene oxides (PPO), as well as block copolymers of PEO and PPO (also known as Pluronics (Registered Trade Mark); (3) cellulose ethers including hydroxypropyl methylcellulose (HPMC), hydroxypropylcellulose (HPC), hydroxyethyl cellulose (HEC), methylcellulose (MC), sodium carboxymethylcellulose (NaCMC); (4) polylactides (PLA), polyglucolides (PGA), copolymers of polylactide and polyglucolide in various proportions (PLGA); (5) poly (sucrose acrylates); (6) polylysine, polyvinylamine, polyethylimine (PEI), polyglutamic acid, polyvinyl alcohol (PVA); copolymers of ethylene and vinyl acetate (pEVA); (7) polyethyleglycol terephthalate, polybutylene terephthalate and copolymers thereof (also known as Locteron [Registered Trade Mark]); Copolymers of PEG and PLGA also known as Re-Gel (Registered Trade Mark); Polyorthoesters also known as Chronomer (Registered Trade Mark); polyanhydrides; copolymers of acrylic acids and esters, or methacrylic acids and esters of various molecular weight and proportion also known as Eudragit (Registered Trade Mark) in particular RL30D, RLPO, RL100, RS30D, RSPO, RS100, NE30D, NM30D, NE40D, L100; copolymers of phthalic acid cellulose and phthallic ester cellulose also known as CAP (Registered Trade Mark); (8) polyvinylpyrrolidone also known as Kollidon (Registered Trade Mark) and copolymers thereof with polyvinyl acetate also known as Kollidon SR (Registered Trade Mark); (9) polymers of natural origin including non ionic, amino, carboxylated and sulfated polysaccharides, optionally chemically modified through partial hydrolysis and/or conjugation of modifiers such as carboxylates or long chain fatty acids (C8-C16), include guar gum; acacia gum, tragacanth gum, carrageenans (both iota and lambda), Linn gum, alginates, scleroglucans, dextrans, chitins and chitosans, pectins, galactomannans including locust bean gum.

In addition, it is frequently found that polymer blends are particularly useful for providing the appropriate release profiles for extended release formulations, for example mixing polymers with hydrophilic and hydrophobic properties, and such polymer blends would include: (1) methyl methacrylates polymers with starch or cellulose polymers; (2) polyacrylic acid-Pluronic-polyacrylic acid block copolymers; (3) multilayer polyelectrolytes using cationic polymers selected from chitosan, polylysine, polyallylamine or poyvinylamine with anionic polymers selected from Carbopols including 971NF, carrageenan, xanthan gum, alginate, hyaluronic acids, Eudragit® including L100 and carboxymethylcellulose; (4) hydrophobic cellulose polymers such as ethylcellulose or Compritol 888 ATO are often mixed with hydrophilic polymers such as HPMC, NaCMC, sodium alginate, xanthan gum or Methocel (Registered Trade Mark); (5) hydrophilic swelling polymer such as HPMC is mixed with a pH dependant polymer such as Eudragit (Registered Trade Mark) L100-55; (6) polymer blends may be crosslinked either by covalent bonds or, particularly for polymers of natural origin, through the addition of polyvalent cations including borate, calcium, magnesium, zinc; (7) natural gums are often used in polymer blends, in particular carrageenans with cellulose ethers, xanthan gum with locust bean gum.

Whilst ternary blends are less common, one example is a blend of non-ionic water soluble polymer Polyox with a swellable high molecular weight cross linked acrylic polymer Carbopol and lactose.

U.S. Pat. No. 5,135,757 and EP 0 642 785 provide matrix compositions for use in extended release formulations. In a particular embodiment of this matrix, the polymer blend comprises xanthan gum and locust bean gum.

U.S. Pat. No. 4,861,598 provides matrix compositions for use in extended release formulations. In general, the compositions make use of higher aliphatic alcohols and acrylic acid resins. In particularly preferred embodiments of this matrix, the polymer comprises the Eudragits RL, RS, S, E30D and L30D. US application 2008/0260815 provides multiparticulate compositions for the matrix described in U.S. Pat. No. 4,861,598 for use in extended release formulations. US application 2004/0116392 also provides multiparticulate compositions for use in extended release formulation.

U.S. Pat. No. 6,033,686 provides matrix compositions for use in extended release formulations. In general, the compositions make use of Kollidon 90F and stearic acid.

Film coatings are contemplated for use with multi unit dosage forms than monolithic tablets. Coatings are selected which include polymer, solvent and a plasticiser, particularly dibutyl sebacate, diethyl phthalate or propylene glycol. Plasticisers may not be necessary when poly(dimethylsiloxane) or other silane elastomers are used.

Particular examples of surface coatings which can provide a hydrogel barrier upon hydration include the cellulose polymers, Eudragit (Registered Trade Mark) polymers and graft copolymers of polyvinyl acetate, polyvinyl alcohol and PEG, also known as Kollicoat (Registered Trade Mark), for example Kollicoat (Registered Trade Mark) SR and Kollicoat (Registered Trade Mark) IR, used with propylene glycol as plasticiser. The properties of this coating are independent of pH.

Polyelectrolyte multilayers (PEM) are one particular example of a film coating which can be optimized to provide an appropriate rate of drug release through a combination of variables including: (1) the selection of positive and negatively charged polyelectrolytes; (2) the number of layers that are deposited; and (3) the molecular weight of the polyelectrolytes used to form the film.

The permeability of PEMs can be responsive to stimuli whereby a change in pH, ionic strength or temperature has the potential to increase change the permeability to particular solutes.

Multilayer tablet formulations are particularly useful for highly soluble drugs. Such dosage forms include a hydrophilic matrix core with one or two semipermeable coatings, which may be implemented as a film or compressed barrier. Typical polymers include cellulose derivatives particularly HPMC, NaCMC, HPC or MC, or natural gums particularly tragacanth or guar gum.

Oral osmotic drug delivery systems make use of a semi-permeable membrane barrier that is significantly more permeable to water than to dissolved drug and other osmotic agents. The drug is combined with one or more polymers and other agents and contained within the membrane barrier, upon oral administration, the process of dissolution of the drug and other osmotic agents provides the driving force for the release of the drug. The drug is effluxed either through a preformed hole of sufficient diameter in the membrane as defined by Poiseuilles law, or the membrane is provided with pores that permit release of the drug.

The rate of dissolution of the drug, the osmotic pressure and the properties of the semi-permeable membrane are features which can be optimized to provide zero order kinetics of drug delivery. For example, it is known that drug release is reduced by increasing the thickness of the semi-permeable membrane, or increasing the polymer content of the matrix relative to drug content.

Elementary Osomotic Pumps (EOPs) comprise a drug-polymer matrix, including additional solutes to increase the osmotic potential. This matrix core is then coated with an semi-permeable surface, through which a tiny hole is bored to provide for water influx and drug release.

In more sophisticated designs, the entire surface is coated with a specialized osmotic membrane, the core comprises a multilayer tablet in which at least one layer is a drug-polymer matrix and one of the layers is constituted to provide the osmotic force. A complete description oral osmotic devices is provided by Malaterre et al. 2007 and Kumar et al. 2007

U.S. Pat. No. 4,612,008 provides a particular oral osmotic drug delivery system.

Osmotic delivery devices typically have a lag time of 30 to 120 minutes before a constant drug release rate is achieved. Early release of drug can be provided by application a drug containing matrix on the outside of the osmotic delivery formulation, optionally coated.

Extended-release formulations within the scope of the present invention can be prepared in accordance with the description provided herein.

### Dosage Levels

In specific embodiments, the optimal therapeutic protocol, and thus the methods of the present invention comprise administration of a total daily dose of 6-TG of between 0.3 mg/kg body weight to 1.5 mg/kg body weight. Suitably the total daily dose of 6-TG is between 0.4 mg/kg body weight and 1 mg/kg body weight, more suitably the total daily dose of 6-TG is between 0.5 mg/kg body weight to 1.0 mg/kg body weight, and even more suitably the total daily dose of 6-TG is between 0.5 mg/kg body weight to 0.8 mg/kg body weight.

It will be appreciated by those skilled in the art that the dosages above, although recited as a daily dosage, may not be administered on a daily basis. For example, a daily dose of 6-TG of 1 mg/kg/body weight may be administered every 2, 3, 4, 5, 6, or 7 days (or otherwise). In an illustrative embodiment, and individual (e.g., adult) of 60 kilograms receiving a daily dose of 6-TG of 1 mg/kg/body weight (i.e., a daily dose of 60 mg) may receive 60 mg/day or may instead receive 120 mg every 2 days, or 180 mg every 3 days, etc. In another illustrative embodiment, an individual (e.g. child) of 20 kilograms may receive the same daily dose of 6-TG (1 mg/kg/body weight) equating to 20 mg/day, or 40 mg/2 days or 60 mg/3 days, etc.

Accordingly, in certain embodiments the composition of the present invention comprising 6-TG comprises between 0.3 mg/kg body weight and 1.5 mg/kg body weight of 6-TG. Suitably the composition comprises between 0.4 mg/kg body weight and 1 mg/kg body weight of 6-TG, more suitably the composition comprises between 0.5 mg/kg body weight and 1.0 mg/kg body weight of 6-TG, and even more suitably the composition comprises between 0.5 mg/kg body weight and 0.8 mg/kg body weight of 6-TG.

The precise dosage of 6-TG in the compositions of the present invention can depend on a variety of factors, such as the intended mode of administration, the disease or condition to be treated, the progression or stage of the disease or condition, and/or the individual to be treated, including the age, gender, height, and/or weight of the individual. It is expected that the precise dosage can be determined by a practitioner in the art. In determining the dosage, the practitioner may evaluate the severity of the disease or condition, or the severity of the symptoms of the disease or condition, the individual's clinical history and responsiveness to previous therapies including any history of prior relapse of the disease or condition. It is expected that the dosage may be readily determined without undue experimentation.

### Formulation and Administration Routes

The compositions of the present invention or the compositions used in the methods of the present invention may be formulated and administered using methods known in the art. Techniques for formulation and administration may be found in “Remington's Pharmaceutical Sciences,” Mack Publishing Co., Easton, Pa., latest edition. Suitable administration routes include enteral routes (including oral, sublingual, buccal, intestinal), and parenteral routes (including intravenous or intraperitoneal injections, or administration by cannulated delivery).

The compositions may comprise a pharmaceutically acceptable carrier and/or diluent, including those that impart the desired consistency, viscosity, texture and appearance.

The compositions may, for example, be formulated readily using a pharmaceutically acceptable carrier and/or diluent, including those well known in the art, into dosages suitable for oral administration, which is also preferred for the practice of the present invention. Such diluents and carriers enable the compositions to be formulated into dosage forms such as tablets, pills, capsules, liquids, gels, syrups, slurries, suspensions and the like, for oral, buccal, or sublingual administration to an individual. Acceptable diluents and carriers are familiar to those skilled in the art and include, but are not restricted to, saline, pyrogen-free or sterile water, sugars, starches, cellulose and its derivatives, malt, gelatine, talc, calcium sulphate, vegetable oils, synthetic oils, polyols, arginic acid, phosphate buffered solutions, and emulsifiers.

Pharmaceutical preparations for enteral use can be obtained by combining the active compounds with solid carriers, optionally grinding a resulting mixture, and processing the mixture of granules, after adding suitable auxiliaries, if desired, to obtain tablets or dragee cores. Suitable carriers are, in particular, fillers such as sugars, including lactose, mannose, mannitol or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any one of the methods of pharmacy but all methods include the step of bringing into associate 6-TG with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids. In addition, stabilisers may be added.

Pharmaceutical formulations for parenteral administration include aqueous solutions of the active compounds in water-soluble form. For injection, the composition may be formulated in aqueous solutions, suitably in physiologically compatible buffers such as Hanks' solution, Ringer's solution, or physiological saline buffer. In such formulations it may be necessary to include a solubilising agent to assist in solubilising the 6-TG.

In some embodiments, the compositions are formulated for constant infusion, or the methods comprise administering the 6-TG by constant infusion.

### Therapeutic Drug Management (“TDM”)

In some embodiments, the method further comprises therapeutic drug monitoring (“TDM”).

Suitably, this method comprises treating a disease or condition responsive to 6-TG comprising a first step comprising administering one or more compositions comprising 6-TG in accordance with the present invention, a second step comprising determining the individual's response to the first administration step, and a third step comprising administering one or more further composition(s) comprising 6-TG where the further compositions are formulated (e.g., with a particular dosage) so as to achieve treatment of the disease or condition based on the individual's response to the first administration.

The individual's response to the first administration may be determined by measuring the levels of white blood cells in the individual, noting the individual's responsiveness to treatment, determining levels of 6-TG in the red blood cells in the individual, or other methods used in the art to determine either the effectiveness of the therapy or the presence of undesirable side-effects.

6-TG at levels that are too high can lead to leukopenia. Accordingly, the second step may comprise measuring the level of white blood cells to determine whether leukopenia is occurring or is at risk of occurring. The white blood cell levels may be measured by any method known in the art. In some embodiments, when the white blood cells measures between 1.0 and 3.5×10⁹/l, the dosage of 6-TG administered in the third step is adjusted to be lower than the dosage administered in the first step, and when the white blood cells measures between 1.0 and 3.5×10⁹/l, the dosage of 6-TG administered in the third step is adjusted to be significantly lower than the dosage administered in the first step, or alternatively 6-TG therapy is halted until the white blood cell count is greater than 1.0, 2.0, 3.0, 3.5, or 4.0×10⁹/l, depending on the value measured in the first step and a target value considered acceptable in the circumstances of the individual being treated.

The second step may comprise obtaining a measurement of 6-TG in the red blood cells. In some embodiments, a measurement of 6-TG in the blood of from about 600 pmol/8×10⁸ red blood cells to about 1800 pmol/8×10⁸ red blood cells is considered acceptable. If the measurement of 6-TG is lower, the next 6-TG dosage is increased. If the measurement of 6-TG is higher, the next 6-TG dosage is decreased. The measurement of 6-TG in the red blood cells may be made in accordance with the method described in Dervieux et al. 1998. Generally, measurement of 6-TG in the red blood cells is imperfect because of the complex metabolism pathways in the body (Duley et al. 2005).

In order that the invention may be readily understood and put into practical effect, particular preferred embodiments will now be described by way of the following non-limiting examples.

### EXAMPLES

### Example 1

### SOS (VOD) is Closely Associated with 6-TG

6-12 week old male or female C57BL/6 mice were gavaged daily with 6-TG (2.5 mg/kg body weight/day) or 6-MP, (≦5 mg/kg body weight/day) methyl-6-MP (≦5 mg/kg body weight/day), or methyl-6-TG (≦2.5 mg/kg body weight/day) for 14 days.

Veno-occlusive disease (or “VOD”) in each mouse was scored in a blinded fashion based on predetermined scoring criteria for sinusoidal endothelial damage (maximum 60), hepatic central vein damage (maximum 60) and inflammatory cell infiltrates using haematoxylin-eosin sections counterstained with vWF for endothelial cells and F4/80 for macrophages (maximum 120). The total possible VOD score was 240.

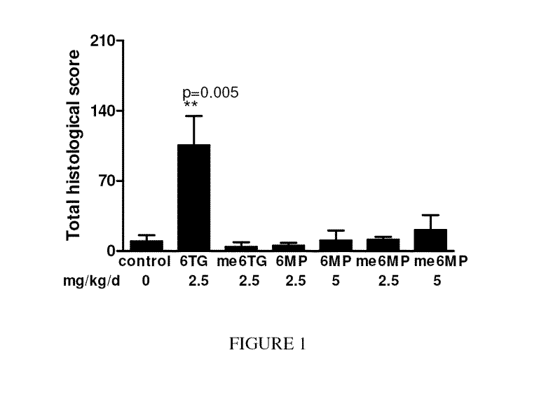

The results are shown in FIG. 1 and demonstrated that SOS (VOD) was only observed in the mice treated with 6-TG. This experiment thus demonstrates that 6-TG, but not 6-MP or their methylated bases causes SOS in the mouse model used.

### Example 2

### HPRT^{-/-} Mouse Model

C57BL/6 mice (n=6) and HPRT^{-/-} C57BL/6 mice (n=6) were administered various doses of 6-TG (0, 0.05, 0.2, 0.5, 1, 2.5 mg/kg body weight) by gavage each day for 14 days.

VOD was scored using the method described in Example 1.

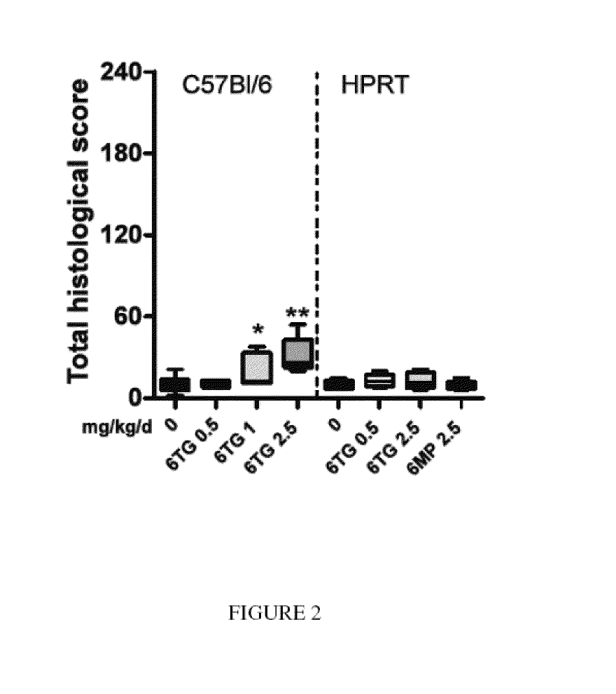

The results are shown in FIG. 2 as box and whisker plots (median, quartiles and range) where * P≦0.05, ** P≦0.01, *** P≦0.005, Mann Whitney non-parametric tests. On the left hand side of the graph in FIG. 2, the results for the C57BL/6 mice are shown. Dosages of 6-TG at 1 mg/kg body weight and above (both 1 mg/kg body weight and 2.5 mg/kg body weight) led to a VOD effect as shown. On the right hand side of the graph in FIG. 2, the results for the HPRT^{-/-} C57BL/6 mice are shown. VOD was shown to be abrogated at all dosage levels, including 1 mg/kg body weight and 2.5 mg/kg body weight.

These results indicate that HPRT^{-/-} knockout mice administered 6-TG do not display SOS found in their wild-type counterparts. The metabolism of 6-TG to 6-TGTP (and/or 6-TGMP or 6-TGDP), the principal active drug, is catalysed by the phosphoribosyltransferase, HPRT. Thus in the absence of HPRT there is no formation of the active drug. These results therefore suggest that it is more likely to be the active drug thioguanosine nucleotides (presumably 6-TGTP itself but also other nucleotide metabolites), and less likely the other metabolites of 6-TG, that is responsible for the undesirable side-effect of SOS associated with 6-TG.

### Example 3

### Intraperitoneal Dosage

Two groups of C57BL/6 mice were administered a large bolus dose of 6-TG (2.5 mg/kg body weight/day) either (1) orally (by gavage) or (2) intraperitoneally for a period of 10 days.

VOD in each mouse was then scored using the method described in Example 1.

As shown in Examples 1 and 2, mice who received 6-TG at a dose of 2.5 mg/kg body weight/day for the 10 day period by oral gavage exhibited VOD. However, VOD was not observed in the second group of mice that received the same amount of 6-TG into the peritoneal cavity.

Intraperitoneal administration avoids the first pass portal circulation, and thus a substantially reduced amount of 6-TG (compared to the oral administration) would have reached the liver cells.

The results of this example thus show that it is the action of a high concentration of the thioguanine nucleotides on the liver cells (off-target cells) which induces the SOS (VOD/NRH) side-effect observed with conventional 6-TG therapy.

### Example 4

### Divided Dose

C57BL/6 mice were administered a total dose of 6-TG (2.5 mg/kg body weight/day) where administration consisted of two separate administrations each day by oral gavage of half the total dose (1.25 mg/kg body weight/twice daily), for a total of 11 days.

VOD in each mouse was then scored using the method described in Example 1, and the results for these mice were compared to the mice who received a single morning dose of 6-TG (2.5 mg/kg) and 0 mg/kg by oral gavage as described in Examples 1, 2 and 3.

The mice receiving the divided dose of 6-TG, despite receiving the same total daily dose of 6-TG as the mice who received the bolus dose, displayed a significantly less VOD score.

### Example 5

### Human Patient

### Divided Dose

In 2003, a female patient (R-LS, d.o.b. June 1975) presented to the inventors with an “exuberant phenotype” of ulcerative colitis. She had previously exhibited life-threatening adverse reactions to AZA, 6-MP and sulfasalazine.

Because treatment with AZA and 6-MP was not an option, she was treated instead with 6-TG using the conventional therapeutic protocol, namely bolus administration of 40 mg per day (R-LS weighs approximately 60 kg) and 5-aminosalicylic acid. After 4 months, R-LS exhibited extremely positive response to the treatment and therefore the 6-TG therapy was halted and long-term treatment continued on a low dose of 5-aminosalicylic acid (maintenance therapy to reduce the chance of relapse). Two years later (July 2005), R-LS exhibited complete symptomatic remission.

In April 2006, R-LS presented again to the inventors with a relapse of ulcerative colitis. Again, she was treated with 6-TG using the conventional therapeutic protocol, namely bolus administration of 40 mg per day and balsalazide. This treatment lasted ˜5-6 months and resulted in a positive response by R-LS. Maintenance therapy to prevent relapse was again prescribed using a lower dose of balsalazide.

R-LS stopped taking the maintenance therapy, and a relapse of her ulcerative colitis occurred in December 2010. The relapse in this instance was severe. Given her clinical history, the inventors were reluctant to prescribe another bolus dose of 6-TG due to the known hepatotoxic side-effects that could occur. Based on their earlier discovery that it is the active drug formed by metabolism of 6-TG (i.e., 6-TGTP) that causes the liver toxicity, the inventors proposed a divided dose of the same treatment prescribed in the two prior instances of relapse.

Accordingly, R-LS was given 60 mg per day 6-TG in a divided dosage (3 separate doses of equal amounts). The doses were given at intervals throughout the day.

The 6-TG was once again effective and after six months, 6-TG treatment was stopped because the inventors were concerned about the possibility of side effects. However, since this time, no side effects have been observed in R-LS.

### Example 6

### Divided Dosing in Disease

This experiment usesa mouse model of spontaneous colitis called Winnie(C57Bl/6 Muc2^{Winnie/Winnie}) due to a single SNP mutation in Muc2 in C57Bl/6 mice. The model resembles ulcerative colitis. The mutation confers an intestinal epithelial cell defect, which causes a Th17 dominant inflammatory response (Heazlewood et al. 2008 and Eri et al. 2011).

Male or female C57Bl/6 Winnie mice were gavaged twice daily for 11-days in an experiment with three groups (6 animals per group):

Group 1: 1.25 mg/kg 6TG evening and morning (total 2.5 mg/kg/d, N=6)

Group 2: vehicle control twice daily (N=6), or

Group 3: 2.5 mg/kg/d 6TG in the morning and vehicle gavage in the afternoon (N=12).

Histological colitis scores from each third of the colon showed significantly improved spontaneous colitis in Winnie mice treated with 6TG 1.25 mg/kg twice daily or 2.5 mg/d gavaged once daily. This together with Example 4 indicates that high dose daily gavage of 6-TG (2.5 mg/kg/d×11 days) prevents development of the spontaneous colitis in Winnie but causes SOS. When 6-TG 2.5 mg/kg/d is delivered to the Winnie mice in a split 1.25 mg/kg b.d dose the spontaneous colitis in the Winnie mice is improved and SOS is largely avoided. Both single full dosing and split dosing were associated with a moderate immunosuppression (as evidenced by reductions in venous blood leukocytes), which is thought to reflect the mechanism of action in the treatment of colitis. There were trends for increased treatment efficacy and immunosuppression with once daily gavage (P=0.07). (This could reflect non-linear first-pass metabolism).

The results of this example thus show that in a situation where colitis is already present that oral split dosing of 6-TG can reduce the magnitude of colonic inflammation but avoid hepatic damage. The split-dose drug-delivery method delivers the drug but avoids high hepatoportal Cmax of 6-TG and its principal active metabolite, the 6-thioguanine nucleotide, in liver. These results enable the inventors to define a therapeutic protocol for 6-TG therapy that avoids high Cₘₐₓ in the portal circulation, while also resulting in immunosuppression and therapeutic efficacy.

### Example 7

### Lower Daily Dose 6TG Over 28 Days Causes Less Immunosuppression, Reduces Spontaneous Colitis and Avoids SOS

This experiment uses Winnie(C57Bl/6 Muc2^{Winnie/Winnie}).

Male or female C57Bl/6 Winnie mice were gavage once daily for 28-days in an experiment with two groups (6 animals per group):

Group 1: 0.5 mg/kg 6TG once daily gavage

Group 2: vehicle control (N=6),

Histological colitis scores from each third of the colon showed significantly improved spontaneous colitis in Winnie mice treated with 6TG 0.5 mg/kg/d. SOS as scored in example 1 did not occur in these animals Immunosuppression was mild in these animals. They did not have myelotoxicity. However, the improvement in colitis was less than for the higher dose over a shorter period of time.

These results enable the inventors to define a therapeutic protocol for 6-TG therapy that avoids high Cₘₐₓ in the portal circulation, while also resulting in therapeutic efficacy and avoiding poor survival resulting from SOS and/or myelotoxicity. The efficacy of 6-TG by mini-osmotic or i.p. delivery is expected to be the same as that by oral gavage and the avoidance of high portal circulation/hepatic Cₘₐₓ is expected to prevent SOS.

### Example 8

### Extended Release Tablets

The following formulations were prepared using SRII 6-Flask Dissolution Test Station (Hanson Research, Chatsworth, Calif., USA). The aim was to produce a three hour extended release tablet of 6-thioguanine (6-TG). The proposed release rate for the tablet was 30±15% in the first hour (in the stomach), 60±15% in the second hour (in the small intestine), and 80±15% in the third hour (in the small intestine).

The materials used in the tablet formulations were as follows:

Hydrroxypropyl methylcellulose (HPMC)

Mannitol

Talc powder

Polyethylene glycol (PEG)

Hydroxypropyl-beta-cyclodextrin (HP-β-CD)

Microcrystalline cellulose (MCC).

The tablets were prepared by direct compression (after mixing of the powders) using a tablet press (Korsch XPI, Germany).

The release rates were determined (USP General Chapter <711> Dissolution, USP Dissolution Apparatus 2-Paddle) in a vessel with a paddle at 75 rpm at 37±0.5° C. Six tablets were placed in an individual vessel which was immersed in 750 mL of hydrochloric acid (HCl, 0.1 N) for one hour. After one hour operation, 200 mL of tribasic sodium phosphate buffer (Na₃PO₄, 0.20 M) that has been equilibrated to 37±0.5° C. was added to the fluid in the vessel. The pH was adjusted to 7.5, if necessary, with 2 N hydrochloric acid or 2 N sodium hydroxide. The operation of adding the buffer and adjusting the pH was completed within 5 minutes. The dissolution apparatus continued to operate for two hours. An aliquot of 1 mL was withdrawn for analysis at 1.2, 2 and 3 hours. Immediately, the amount withdrawn was replaced with fresh buffer solution (pH 7.5 buffer solution).

### Formulation 1

### Formulation 1

| Ingredients | Amount (mg) |
|---|---|
| 6-TG | 5 |
| HPMC(K4M-CR) | 12.5 |
| HPMC(K100-CR) | 15 |
| HP-β-CD | 50 |
| PEG 6000 | 50 |
| Talc Powder | 2.5 |
| Total | 135 |

### Release Rate of Formulation 1

| Time | 6-TG Release | |
|---|---|---|
| (h) | Percentage (%) | SD |
| 1 | 26.30 | 16.5 |
| 2 | 62.76 | 12.39 |
| 3 | 78.86 | 6.77 |

HP-β-CD was employed for improving the solubility of 6-TG and proved to be particularly useful. When HP-β-CD is used it may be advantageous to keep the mixture at elevated temperature for a period of time (for example at about 65° C. overnight) before tablet preparation, using careful humidity control, since high humidity interferes with tablet formation. Formulation 1 achieved the expected release rate, but the reproducibility of this formulation was not always good. This may be due to the low melting point (58.71° C.) and poor flowability of PEG6000.

### Formulation 2

### Formulation 2

| Ingredients | Amount (mg) |
|---|---|
| 6-TG | 5 |
| HPMC(K4M) | 6 |
| HPMC(K100) | 7.5 |
| Mannitol | 39 |
| HP-β-CD | 40 |
| Talc powder | 2.5 |
| Total | 100 |

### Release Rate of Formulation 2

| Time | 6-TG Release | |
|---|---|---|
| (h) | Percentage (%) | SD |
| 1.2 | 34.15 | 7.26 |
| 2 | 56.29 | 12.18 |
| 3 | 76.57 | 6.3 |

Mannitol replaced PEG and lactose in formulation 2. The release rate of this formulation is good and the reproducibility is acceptable.

### Example 9

75 mg of 6-TG was dissolved in 7.5 mL 0.1 mol NaOH, 600 mg of HP-β-CDs was added and the mixture freeze dried for about 40 hours. The freeze dried powder was mixed with HPMC to produce a tablet. The compressibility of mixture became worse after the freeze drying step. The release rates were determined as described above in Example 7. The results are shown in the table below:

| Time | Dissolution | |
|---|---|---|
| (h) | percentage | SD |
| 1.2 | 13.01 | 5.71 |
| 2 | 27.71 | 6.53 |
| 3 | 44.24 | 8.57 |
| 4 | 48.51 | 11.62 |

The portal circulation dose exposure is believed to be replicable using extended-release formulations of 6-TG or divided dose administration regimes for 6-TG in humans.

Australian Application No. 2011902301, from which priority is claimed, is hereby incorporated herein by reference in its entirety. All of the above U.S. patents, U.S. patent application publications, U.S. patent applications, foreign patents, foreign patent applications and non-patent publications referred to in this specification and/or listed in the Application Data, Sheet, are incorporated herein by reference.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims. Many modifications will be apparent to those skilled in the art without departing from the scope of the present invention.

### BIBLIOGRAPHY

Ansari A, Arenas M, Greenfield S M, Morris D, Lindsay J, Gilshenan K, Smith M, Lewic C, Marinaki A, Duley J, Sanderson J, “Prospective evaluation of the pharmacogenetics of azathioprine in the treatment of inflammatory bowel disease” 15 Oct. 2008 Aliment Pharmacol Ther. 28(8): 973-983.
Dervieux T, Boulieu R, “Simultaneous determination of 6-thioguanine and methyl 6-mercaptopurine nucleotides of azathioprine in red blood cells by HPLC” March 1998 Clin Chem 44(3): 551-555.
Dubinsky M C, Yang H, Hassard P V, et al. “6-MP metabolite profiles provide a biochemical explanation for 6-MP resistance in patients with IBD” 2002 Gastroenterology 122: 904-915.
Duley J A, Florin T H J, “Thiopurine Therapies. Problems, Complexities, and Progress With Monitoring Thioguanine Nucleotides” 2005 Ther Drug Monit 27: 647-654.
Eri R D, Adams R J, Tran T V, Tong H, Das I, Roche D K, Oancea I, Png C W, Jeffery P L, Radford-Smith G L, Cook M C, Florin T H, McGuckin M A, “An intestinal epithelial defect conferring ER stress results in inflammation involving both innate and adaptive immunity” May 2011 Mucosal Immunol. 4(3): 354-364.
Heazlewood C K, Cook M C, Eri R, Price G R, Tauro S B, Taupin D, Thornton D J, Png C W, Crockford T L, Cornall R J, Adams R, Kato M, Nelms K A, Hong N A, Florin T H, Goodnow C C, McGuckin M A, “Aberrant mucin assembly in mice causes endoplasmic retriculum stress and spontaneous inflammation resembling ulcerative colitis” 4 Mar. 2008 PLoS Med. 5(3): e54.
Hindorf U, Lindqvist M, Hildebrand H, Fagerberg U, Almer S, “Adverse events leading to modification of therapy in a large cohort of patients with inflammatory bowel disease” 15 Jul. 2006 Aliment Pharmacol Ther. 24(2): 331-342.
Kumar P, Mishra B, “An overview of recent patent on oral osmotic drug delivery systems” 2007 Recent Patent Drug Del Form 1:236-255.
Malaterre V, Ogorka J, Loggia N, Gurny R, “Oral osmotically driven systems: 30 years of development and clinical use” 2007 Eur J Pharm Biopharm 73:311-323.
McGovern J, Travis S, Duley J, Dalton H, “Azathioprine intolerance in patients with IBD may be imidazole-related and is independent of TMPT activity” 2002 Gastroenterology 122: 838-839.
Seinen M L, van Asseldonk D P, Mulder C J J, de Boer N K H, “Dosing 6-Thioguanine in Inflammatory Bowel Disease: Expert-Based Guidelines for Daily Practice” September 2010 J Gastrointestin Liver Dis 19(3): 291-294.
Stork L, Matloub Y, Broxson E, et al. “Oral 6-mercaptopurine versus oral 6-thioguanine and veno-occlusive disease in children with standard-risk acute lymphoblastic leukemia: report of the Children's Oncology Group CCG-1952 clinical trial” 2010 Blood 115:2740-2748.

What is claimed is:

1. A composition comprising an extended-release formulation of 6-TG.

2. A pharmaceutical composition comprising 6-TG and a pharmaceutically acceptable carrier suitable for providing extended release of 6-TG.

3. The composition of claim 1, wherein the composition is formulated to provide a dose of 6-TG of between 0.3 mg/kg body weight/day to 1.5 mg/kg body weight/day.

4. The composition of claim 1 or claim 2, wherein the composition is formulated for oral administration.

5. The composition of claim 1 or claim 2, wherein the composition is for treating a disease or condition responsive to 6-TG.

6. A method for treating a disease or condition that responds to 6-TG in an individual in need thereof, the method comprising administering to the individual a total dose of 6-TG, wherein the method avoids or at least reduces the 6-TG side-effects observed when the total dose of 6-TG administered orally in bolus form.

7. The method of claim 6, wherein the total dose of 6-TG comprises between about 0.3 mg/kg body weight/day to 1.5 mg/kg body weight/day.

8. The method of claim 6 or claim 7, wherein the method comprises oral intraperitoneal, buccal, sublingual, transdermal or intranasal administration of the 6-TG,

9. The method of claim 6 or claim 7, wherein the method comprises administering a composition comprising an extended-release formulation of 6-TG.

10. The method of claim 9, comprising administering a composition of claim 1 or claim 2.

11. The method of claim 6 or claim 7, wherein the method comprises an administration regime that comprises administering more than one composition comprising 6-TG, wherein the total dose of 6-TG is divided into the compositions, wherein the compositions are not administered at the same time.

12. The method of claim 6 or claim 7, further comprising administering an additional active agent for treating a disease or condition responsive to 6-TG.
